(19) United States
(12) Patent Application Publication
(10) Pub. No.: US 2003/0207946 A1
(43) Pub. Date: Nov. 6, 2003

(54) Novel parenteral composition comprising propofol

(75) Inventors: Jong Woo Park, Kyunggi-do (KR); Sang-Cheol Chi, Kyunggi-do (KR); Eun-Seok Park, Kyunggi-do (KR); Kyu-Hyun Lee, Kyunggi-do (KR)

Correspondence Address: GREENBLUM & BERNSTEIN, P.L.C., 1950 ROLAND CLARKE PLACE, RESTON, VA, 20191, US

(73) Assignee: FDL, Inc., Kyunggi-do (KR)

(21) Appl. No.: 10/295,863

(22) Filed: Nov. 18, 2002

(30) Foreign Application Priority Data
May 2, 2002 (KR) 2002-24058

(51) Int. Cl. A61K 31/4172; A61K 31/05; A61K 31/198

(52) U.S. Cl. 514/731; 514/400; 514/564; 514/565

(57) ABSTRACT

The present invention relates to a novel parenteral composition comprising propofol and electrokinetic stabilizer.

The electrokinetic stabilizer maintains the zeta potential of the propofol injection at a higher value than the critical zeta potential as absolute value. The electrokinetic stabilizer is pharmaceutically acceptable and injectable, and is selected from the group consisting of a basic amino acid such as lysine, arginine or histidine; a basic compound or a salt such as monoethanolamine, diethanolamine, sodium carbonate, sodium bicarbonate, tromethamine or sodium phosphate; or a mixture thereof.

Even though a large amount of lidocaine is admixed to the propofol injection of this invention to reduce the pain on injection of propofol, or a relatively long time elapsed after admixing, the propofol composition of this invention maintains physicochemical stability of the preparation and, thus, can be used as a painless, effective and safe intravenous anesthetic without severe adverse effect such as pulmonary embolism.

### BACKGROUND OF THE INVENTION

The present invention relates to a novel parenteral composition comprising propofol.

Propofol, (2,6-diisopropylphenol), is a parenteral anesthetic which has hypnotic properties and can be used to induce and maintain general anesthesia and sedation. Its onset time for reaction and recovery from anesthesia is fast, since it acts quickly on the central nerve system after passing through the blood brain barrier easily due to its high lipophilicity. Propofol is poorly water-soluble, and therefore is generally formulated as a lipid emulsion.

However, in a clinical trial, more than 92% of patients experienced severe pain with the injection of the above propofol in conventional lipid emulsion (Kelement, W., Brit. J. Anaesth., 67, 281-284, 1991). Although the mechanism of pain on injection of propofol remains unclear, kinin cascade theory is most widely accepted. It is suspected that injection of propofol could result in the release of certain mediators such as kininogen, which is thought to mediate or facilitate the pain process.

The attempts for the reduction of pain on injection of propofol are; 1) the addition of local anesthetic agent such as lidocaine or prolicaine to the propofol emulsion 2) the administration of propofol via antecubital fossa vein 3) the administration of propofol at low temperature, for example, 4° C. 4) the premedication of alfentanyl, thiopental or metoclopamide. Among them, the addition of lidocaine to propofol emulsion is the most widely used method clinically. Once injected, lidocaine reduces the pain by acting as the local anesthetic on the vein wall of injection site or the block to a pain mediator.

It is well known that the physicochemical stability of lipid emulsion depends on the electrical characteristic of the surface of oil globules. It has been reported that oil globules of lipid emulsion have negative charge owing to anionic surfactant, such as lecithin, surrounding them. The electrostatic repulsive forces between oil globules due to the charge contribute the stability of lipid emulsion (Washington C., Int. J Pharm., 54, 191-197, 1989; Washington C., Int. J Pharm., 87, 167-174, 1992; Washington C., Int. J Pharm., 66, 1-21, 1990).

However, lidocaine, which may be divalent cationic in water when added to propofol emulsion, can neutralize the anionic charge on the surface of oil globules. This may reduce the electrostatic repulsive forces of oil globules. It can be easily quantified using zeta potential, which is the potential at the shear plane of oil globules. When the zeta potential of propofol emulsion is decreased, oil globules may coalesce, to form larger globules, and eventually phase separation occurs (Lilley E. M. M., Anaesthesia, 51, 815-818, 1996; Maska Y., Anesth. Analog., 90, 989-992, 2000). Also, it is reported that oil globules in the emulsion, larger than 5.0 μm, can cause pulmonary embolism, which can induce fatal results (Driscoll D. F., Am. J. Health-Syst. Pharm., 52, 623-634, 1995; Koster V. S., Int. J Pharm., 134, 235-238, 1996).

When a small amount of lidocaine is admixed to propofol emulsion or propofol emulsion is injected to a patient immediately after lidocaine is admixed, the globule size of propofol emulsion might not be increased significantly. However, in order to reduce the pain on injection, a large amount of lidocaine is necessary, up to 30 mg, occasionally 40 mg or 50 mg, based on 200 mg of propofol, depending on injection site, injection rate, administration situation, size of cannula, race and patient condition (Gajraj N. M., J Clin. Anesth., 8, 575-577, 1996; Ho C.-M., J Clin. Anesth., 11, 296-300, 1999). Also, a relatively long period, more than a few hours, may be passed due to the injection schedule in a hospital.

Therefore, it is necessary to develop an appropriate formulation of propofol emulsion in order to maintain the stability of propofol emulsion during an appropriate time interval adequate for injection and, thus, prevent fatal adverse effects which can occur during clinical application even though a large amount of lidocaine is added or a relatively long time elapses until injection after the admixing of lidocaine to propofol emulsion.

### SUMMARY OF THE INVENTION

The present invention provides a novel parenteral composition comprising propofol and the electrokinetic stabilizer to maintain the physicochemical stability of propofol injection even though a large amount of lidocaine is added or a relatively long time elapses until injection after the admixing of lidocaine to propofol emulsion.

### DETAILED DESCRIPTION OF THE INVENTION

The present invention provides a novel parenteral composition comprising propofol and electrokinetic stabilizer. The electrokinetic stabilizers are compounds or mixture of compounds that, by their presence in colloidal systems, resist changes in electrokinetic properties upon the addition of small quantities of additives which disturbs the electrokinetic balance of colloidal systems.

The composition of the present invention comprises propofol, water- immiscible solvent, anionic surfactant, tonicity agent, electrokinetic stabilizer and water.

Colloidal systems, such as emulsion and suspension, have been commonly used as delivery systems for some drugs. Such colloidal systems are used after the drug is put into a dispersed phase (solid or liquid) and, then, suspended or dispersed in a dispersion medium. The stability of the colloidal system is affected by the characteristics of the interface between the dispersed phase and the dispersion medium.

Generally, in the colloidal systems, the physical stability of dispersed phase is mainly affected by van der Waals forces (attractive forces between dispersed phases) and electrostatic forces (repulsive forces between dispersed phases). The electrostatic force can be expressed as zeta potential with a negative or positive value. The electrostatic repulsive forces become larger as the absolute value of the zeta potential increases.

The colloidal system is used clinically, sometimes, after some additives are added to it. However, these additives can disturb the electrokinetic balance of the system and consequently render the system unstable. Especially, multivalent substances change the electrokinetic characteristics of the interface, since they bind directly to the globule surface, chemically or electrically. It commonly decreases zeta potential and eventually induces charge reversal, when a large amount of multivalent substances added.

At a certain low absolute value of zeta potential, oil globules begin to coalesce. The zeta potential at this point is called as the critical zeta potential. It depends on the composition of the colloidal system and the characteristics of the additives. At the absolute value of zeta potential lower than the critical zeta potential, globules coalesce and finally, the phase separation can occur. Electrokinetic stabilizer is used to prevent such phenomena.

The composition of the present invention is in detail hereinafter.

The composition of the present invention comprises 1.0 to 5.0% by weight of propofol, 1.0 to 30.0% by weight of water-immiscible solvent, 0.2 to 2.0% by weight of anionic surfactant, 0.1 to 3.0% by weight of tonicity agent, 0.005 to 5.0% by weight of electrokinetic stabilizer and water.

In the composition of the present invention, the electrokinetic stabilizer is present in an amount from 0.005 to 5.0% by weight, preferably from 0.01 to 0.5% by weight. At the amount lower than 0.005% by weight, it is difficult to maintain the maximum diameter of globule (D99.99) less than 5.0 μm. At the amount higher than 5.0% by weight, it is difficult to prepare the emulsion due to the increased viscosity of the vehicle and, sometimes, the clinical adverse effect such as hemolysis occurs. The electrokinetic stabilizer is pharmaceutically acceptable and injectable, and is selected from the group consisting of a basic amino acid such as lysine, arginine or histidine; a basic compound or a salt such as monoethanolamine, diethanolamine, sodium carbonate, sodium bicarbonate, tromethamine or sodium phosphate; or a mixture thereof.

In the composition of the present invention, water-immiscible solvent is present in an amount from 1.0 to 30.0% by weight, and is selected from the group consisting of a vegetable oil such as soybean oil, safflower oil, cottonseed oil, corn oil, sunflower oil, peanut oil, castor oil or olive oil; an ester of a medium chain fatty acid; an ester of a long chain fatty acid; or a mixture thereof.

In the composition of the present invention, anionic surfactant is present in an amount from 0.2 to 2.0% by weight, and is selected from the group consisting of a phospholipid such as egg lecithin or soybean lecithin; its derivatives such as phosphatidylcholine, phosphatidylethanolamine, phosphatidylinositol, phosphatidylserine, sphingomyelin, cardiolipin, sulfatide or phosphatidic acid; or a mixture thereof.

In the composition of the present invention, tonicity agent is present in an amount from 0.1 to 3.0% by weight, and is selected from glycerin, mannitol or sucrose; or a mixture thereof.

Additionally, the composition of the present invention can comprise nonionic surfactant, which is selected from polysorbate, poloxamer or sorbitan fatty acid esters; or a mixture thereof.

The conventional propofol emulsions comprising water-immiscible solvent, anionic surfactant, tonicity agent and water have the zeta potential ranges from about −50 to about −30 mV, the pH ranges from about 6.0 to about 8.5, and the maximum diameter of globule (D99.99) less than 1.0 μm. In these products, oil globules have the tendency not to coalesce each other due to the electrostatic repulsive forces of the oil globules surrounded by the anionic surfactant such as lecithin.

When lidocaine, a cationic compound, is added to propofol emulsion, it decreases the negatively charged surface of oil globules in propofol emulsion. When the amount of lidocaine added is increased, the zeta potential of the emulsion passes the critical zeta potential and also 0 mV (point of zero charge), and, eventually, induces slight charge reversal. The pH of the system is also changed to 5.5-6.0.

The decreased repulsive forces between oil globules in the emulsion result in relative increase of attractive forces, which increases the maximum diameter of globules (D99.99) to 3-tens μm. The globule size of the emulsion also increases as time elapses after the admixing lidocaine to the propofol emulsion, which may consequently result in phase separation of emulsion. These preparations may cause severe and fatal side effects such as pulmonary embolism, when injected to a patient.

However, the composition of the present invention comprises pharmaceutically acceptable electrokinetic stabilizer, which maintains the maximum diameter of globules below the injectable criteria, because the electrokinetic stabilizer keeps the balance between the attractive force and the repulsive force of oil globules by preventing the interaction of lidocaine with oil globules in propofol emulsion.

In the composition of the present invention, the oil globules in propofol emulsion do not exceed the clinically acceptable maximum globule size by maintaining the absolute value of zeta potential at higher than critical zeta potential and its pH above 6.0, even though the amount of lidocaine used was increased more than 50 mg which is the clinically acceptable maximum amount for 200 mg of propofol, or a relatively long time elapsed after the addition of lidocaine to propofol emulsion.

Additionally, pharmaceutically acceptable and injectable additives such as antioxidant, buffer and bacteriostatic agent can be added to the composition, if necessary, besides the components cited in the above.

The composition of the present invention is used for the intravenous administration, and it is desirable to administer propofol at the dose of 1.5-2.5 mg/kg for the induction of general narcosis and 4-12 mg/kg/hr for the maintenance of general narcosis, even though it varies with the patient's age, weight, general health condition, gender, diet, administration time and therapy period.

The present invention is more specially explained by the following examples. However, it should be understood that the scope of the present invention is not limited by the examples in any manner.

### EXAMPLE 1

### Preparation of Propofol Injection Containing 0.05% (w/v) Lysine

| | | |
|---|---|---|
| propofol | 3.0 | g |
| soybean oil | 30.0 | g |
| lecithin | 3.6 | g |
| glycerin | 6.75 | g |
| lysine | 0.15 | g |
| water for injection | to 300 | ml |

1) Preparation of Oil Phase

3.0 g of propofol was added to 30.0 g of soybean oil, and the mixture was stirred at 60-85° C. until completely dissolved.

2) Preparation of Aqueous Phase

3.6 g of lecithin was added to an appropriate amount of water for injection, and mixed thoroughly. Then, 6.75 g of glycerin and 0.15 g of lysine were added to the dispersion and heated to 60-85° C. until completely dissolved.

3) Preparation of Propofol Injection

After oil phase was added into the aqueous phase, water for injection was added to make 300 ml, and a coarse emulsion was prepared using a homogenizer (Ultra Turrax, T18/10 S7, IKA, Germany) by agitating at 12000 rpm for 3 min. at 60° C. Then, it was passed through Microfluidizer (M110S, Microfluidic, USA) 5 times at the pressure of 20,000 psi to make fine emulsion.

All the process was conducted under nitrogen atmosphere to prevent the oxidation and degradation of oil and propofol during the preparation

The obtained emulsion was filtered through a 0.45 μm filter and filled into a glass vial under nitrogen atmosphere. Then, it was sealed and autoclaved at 121° C., for 15 min.

### EXAMPLE 2

### Preparation of Propofol Injection Containing 0.2% (w/v) Lysine

| | | |
|---|---|---|
| propofol | 3.0 | g |
| soybean oil | 30.0 | g |
| lecithin | 3.6 | g |
| glycerin | 6.75 | g |
| lysine | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of lysine.

3) Preparation of Propofol Injection

Propofol injection was prepared as in the Example 1-3), described above.

### EXAMPLE 3

### Preparation of Propofol Injection Containing 0.2% (w/v) Arginine

| | | |
|---|---|---|
| propofol | 3.0 | g |
| soybean oil | 30.0 | g |
| lecithin | 3.6 | g |
| glycerin | 6.75 | g |
| arginine | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of arginine.

3) Preparation of Propofol Injection

Propofol injection was prepared as in the Example 1-3), described above.

### EXAMPLE 4

### The Preparation of Propofol Injection Containing 0.2% (w/v) Histidine

| | | |
|---|---|---|
| propofol | 3.0 | g |
| soybean oil | 30.0 | g |
| lecithin | 3.6 | g |
| glycerin | 6.75 | g |
| histidine | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of histidine.

3) Preparation of Propofol Injection

Propofol injection was prepared as in the Example 1-3), described above.

### EXAMPLE 5

### Preparation of Propofol Injection Containing 0.1% (w/v) Diethanolamine

| | | |
|---|---|---|
| propofol | 3.0 | g |
| soybean oil | 30.0 | g |
| lecithin | 3.6 | g |
| glycerin | 6.75 | g |
| diethanolamine | 0.3 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.3 g of diethanolamine.

3) Preparation of Propofol Injection

Propofol injection was prepared as in the Example 1-3), described above.

### EXAMPLE 6

### Preparation of Propofol Injection Containing 0.1% (w/v) Sodium Carbonate

| | | |
|---|---|---|
| propofol | 3.0 | g |
| soybean oil | 30.0 | g |
| lecithin | 3.6 | g |
| glycerin | 6.75 | g |
| sodium carbonate | 0.03 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.03 g of sodium carbonate.

3) Preparation of Propofol Injection

Propofol injection was prepared as in the Example 1-3), described above.

### EXAMPLE 7

### Preparation of Propofol Injection Containing 0.5% (w/v) Tromethamine

| | | |
|---|---|---|
| propofol | 3.0 | g |
| soybean oil | 30.0 | g |
| lecithin | 3.6 | g |
| glycerin | 6.75 | g |
| tromethamine | 1.5 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 1.5 g of tromethamine.

3) Preparation of Propofol Injection

Propofol injection was prepared as in the Example 1-3), described above.

### COMPARATIVE EXAMPLE 1

### Preparation of Propofol Injection Containing 0.2% (w/v) Glutamic Acid

| | | |
|---|---|---|
| propofol | 3.0 | g |
| soybean oil | 30.0 | g |
| lecithin | 3.6 | g |
| glycerin | 6.75 | g |
| glutamic acid | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of glutamic acid.

3) Preparation of Propofol Injection

Propofol injection was prepared as in the Example 1-3), described above.

### COMPARATIVE EXAMPLE 2

### Preparation of Propofol Injection Containing 0.2% (w/v) Aspartic Acid

| | | |
|---|---|---|
| propofol | 3.0 | g |
| soybean oil | 30.0 | g |
| lecithin | 3.6 | g |
| glycerin | 6.75 | g |
| aspartic acid | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of aspartic acid.

3) Preparation of Propofol Injection

Propofol injection was prepared as in the Example 1-3), described above.

### COMPARATIVE EXAMPLE 3

### Preparation of Propofol Injection Containing 0.2% (w/v) Sodium Citrate

| | | |
|---|---|---|
| propofol | 3.0 | g |
| soybean oil | 30.0 | g |
| lecithin | 3.6 | g |
| glycerin | 6.75 | g |
| sodium citrate | 0.6 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 0.6 g of sodium citrate.

3) Preparation of Propofol Injection

Propofol injection was prepared as in the Example 1-3), described above.

### COMPARATIVE EXAMPLE 4

### Preparation of Propofol Injection Containing 0.5% (w/v) Isoleucine

| | | |
|---|---|---|
| propofol | 3.0 | g |
| soybean oil | 30.0 | g |
| lecithin | 3.6 | g |
| glycerin | 6.75 | g |
| isoleucine | 1.5 | g |
| water for injection | to 300 | ml |

1) Preparation of Oil Phase

Oil phase was prepared as in the Example 1-1), described above.

2) Preparation of Aqueous Phase

Aqueous phase was prepared as in the Example 1-2), described above, except using 1.5 g of isoleucine.

3) Preparation of Propofol Injection

Propofol injection was prepared as in the Example 1-3), described above.

### TEST EXAMPLE 1

### Change of Zeta Potential when Lidocaine was Added to Propofol Injection of the Present Invention

The change of zeta potential was observed after 10, 20, 30, 40 and 50 mg of lidocaine (based on 200 mg of propofol) were added to the injection of the Examples 1-6, the Comparative Examples 1-4 and the commercial product (DIPRIVAN®, AstraZeneca, UK).

Zeta potential was measured using AcoustoSizer (Colloidal Dynamic, Aus). Before the measurement, the apparatus was calibrated using the standard solution. After 200 ml of propofol emulsion containing 10 mg/ml of propofol was poured into the sample container of the apparatus and each corresponding amount of lidocaine was added at the scheduled interval and mixed thoroughly by concentration titration method. Sample was passed through the measuring cell and the zeta potential was measured.

The result of zeta potential measurement is presented in Table 1.

TABLE 1
(Unit: mV)

| | Amount of lidocaine added | | | | | |
|---|---|---|---|---|---|---|
| | (mg; based on 200 mg/20 Ml of propofol) | | | | | |
| | 0 | 10 | 20 | 30 | 40 | 50 |
| Example 1 | −72.3 | −51.1 | −26.4 | −14.8 | −7.6 | −4.6 |
| Example 2 | −67.1 | −60.7 | −56.2 | −49.7 | −42.6 | −33.6 |
| Example 3 | −74.8 | −66.0 | −59.7 | −54.1 | −47.4 | −40.2 |
| Example 4 | −49.4 | −29.8 | −19.4 | −12.8 | −6.6 | −4.7 |
| Example 5 | −71.1 | −65.0 | −59.3 | −54.8 | −50.8 | −47.4 |
| Example 6 | −43.7 | −42.4 | −41.0 | −39.4 | −38.4 | −38.7 |
| Comparative | −13.7 | −6.6 | −2.7 | 1.0 | 2.6 | 3.7 |
| Example 1 | | | | | | |
| Comparative | −9.7 | −4.5 | −0.1 | 1.7 | 3.7 | 4.5 |
| Example 2 | | | | | | |
| Comparative | −24.0 | −12.8 | −7.1 | −3.6 | −1.3 | −0.6 |
| Example 3 | | | | | | |
| Comparative | −44.9 | −17.5 | −9.2 | −4.2 | −1.2 | −0.5 |
| Example 4 | | | | | | |
| DIPRIVAN ® | −54.5 | −16.1 | −7.6 | −3.4 | −0.2 | 2.3 |

As shown in Table 1, the absolute values of zeta potential of propofol injection prepared as the Examples 1-6 were kept above the critical zeta potential, 4.5 mV, even though lidocaine was added up to 50 mg. On the other hand, the absolute values of zeta potential were changed to less than the critical zeta potential value, 4.5 mV, in the Comparative Examples 1-2. Also, they were changed to less than the critical zeta potential, 3.5 mV, in the Comparative Examples 3-4 and the commercial product (DIPRIVAN®, AstraZeneca, UK). Therefore, it is revealed that the absolute value of zeta potential of the composition of the present invention was maintained at higher value than the critical zeta potential, even though a large amount of lidocaine was added.

### TEST EXAMPLE 2

### Change of pH when Lidocaine was Added to Propofol Injection of the Present Invention

The change of pH of injection was observed after 10, 20, 30, 40 and 50 mg of lidocaine (based on 200 mg of propofol) were added to the injection of the Examples 1-7, the Comparative Examples 1-4 and the commercial product (DIPRIVAN®, AstraZeneca, UK).

The result of the pH measurement is presented in Table 2.

TABLE 2

| | Amount of lidocaine added | | | | | |
|---|---|---|---|---|---|---|
| | (mg; based on 200 mg/20 Ml of propofol) | | | | | |
| | 0 | 10 | 20 | 30 | 40 | 50 |
| Example 1 | 8.84 | 7.94 | 7.14 | 6.84 | 6.67 | 6.57 |
| Example 2 | 9.37 | 9.16 | 8.93 | 8.68 | 8.39 | 8.05 |
| Example 3 | 9.80 | 9.50 | 9.25 | 9.02 | 8.76 | 8.45 |
| Example 4 | 7.48 | 6.93 | 6.74 | 6.66 | 6.62 | 6.58 |
| Example 5 | 9.77 | 9.52 | 9.33 | 9.19 | 9.06 | 8.94 |
| Example 6 | 8.81 | 8.64 | 8.48 | 8.33 | 8.17 | 8.01 |
| Example 7 | 9.64 | 9.20 | 8.96 | 8.76 | 8.68 | 8.60 |
| Comparative | 3.14 | 3.18 | 3.22 | 3.24 | 3.29 | 3.27 |
| Example 1 | | | | | | |
| Comparative | 2.89 | 2.94 | 2.97 | 2.99 | 3.01 | 3.03 |
| Example 2 | | | | | | |
| Comparative | 7.51 | 6.91 | 6.69 | 6.58 | 6.48 | 6.43 |
| Example 3 | | | | | | |
| Comparative | 7.38 | 6.30 | 6.06 | 5.95 | 5.90 | 5.87 |
| Example 4 | | | | | | |
| DIPRIVAN ® | 7.60 | 6.49 | 6.21 | 6.07 | 5.98 | 5.92 |

As shown in Table 2, the pH of propofol injections of the Examples 1-7 were maintained at 6.0-9.0, even tough lidocaine was added up to 50 mg. On the other hand, the pH of the Comparative Examples 1-2 was became low, 2.0-3.5, regardless of the addition of lidocaine. The pH of the Comparative Example 4 and the commercial product (DIPRIVAN®, AstraZeneca, UK) was decreased to lower than 6.0 when more than 30 mg of lidocaine was added to the propofol emulsion.

Therefore, it is revealed that the pH of the composition of the present invention were maintained at higher than 6.0 compared to the Comparative Examples and the commercial product (DIPRIVAN®, AstraZenaca, UK), even though a large amount of lidocaine was added.

### TEST EXAMPLE 3

### Change of Globule Size when Lidocaine was Added to Propofol Injection of the Present Invention

The size of oil globules was measured at 6 hours after 10, 20, 30, 40 and 50 mg of lidocaine (based on 200 mg of propofol) were added to propofol injection of the Examples 1-7, the Comparative Examples 1-4 and the commercial product (DIPRIVAN®, AstraZenaca, UK). The globule size was measured using MasterSizer X (Malvern, UK) employing laser diffraction method.

The measurement was conducted using 45 mm lens, MS15 wet sample injection apparatus, deionized water as dilution medium, and 2NAD(1.33, 1,456+i0.0000) as presentation mode. The maximum diameter of globule (D99.99) was obtained from the measurement.

The result of globule size is presented in the Table 3.

TABLE 3
(Unit: μm)

| | Amount of lidocaine added | | | | | |
|---|---|---|---|---|---|---|
| | (mg; based on 200 mg/20 Ml of propofol) | | | | | |
| | 0 | 10 | 20 | 30 | 40 | 50 |
| Example 1 | 0.97 | 0.92 | 2.81 | 2.86 | 2.95 | 3.40 |
| Example 2 | 0.91 | 0.90 | 0.89 | 0.92 | 0.90 | 2.89 |
| Example 3 | 0.97 | 0.92 | 2.89 | 2.87 | 2.81 | 3.47 |
| Example 4 | 0.97 | 0.92 | 2.81 | 2.82 | 2.82 | 2.83 |
| Example 5 | 0.96 | 0.97 | 0.97 | 0.97 | 0.97 | 0.97 |
| Example 6 | 0.97 | 0.97 | 0.97 | 2.80 | 2.79 | 2.82 |
| Example 7 | 0.97 | 0.96 | 0.97 | 0.97 | 0.97 | 0.97 |
| Comparative | 0.90 | 3.20 | 69.6 | 79.99 | 79.96 | 21.17 |
| Example 1 | | | | | | |
| Comparative | 0.92 | 7.79 | 79.97 | 79.96 | 60.62 | 6.90 |
| Example 2 | | | | | | |
| Comparative | 0.97 | 2.83 | 2.86 | 3.29 | 45.67 | 74.75 |
| Example 3 | | | | | | |
| Comparative | 0.94 | 3.32 | 2.88 | 3.03 | 79.68 | 79.98 |
| Example 4 | | | | | | |
| DIPRIVAN ® | 0.96 | 3.05 | 2.88 | 51.76 | 79.92 | 79.92 |

As shown in Table 3, the globule size of propofol injection of the Examples 1-7 did not exceed 3.5 μm, even though lidocaine was added up to 50 mg.

On the other hand, the globule sizes of the Comparative Examples 1-4 and the commercial product (DIPRIVAN®, AstraZenaca, UK) were increased significantly even a small amount of lidocaine was added.

Therefore, the maximum diameter of globule size of the composition of the present invention was maintained less than 5.0 μm compared to the Comparative Examples and the commercial product (DIPRIVAN®, AstraZenaca, UK), even at 6 hours after a large amount of lidocaine was added.

[Industrial Applicability]

In the present invention, the effect of the present invention is that the composition of the present invention can be used safely for the intravenous injection of propofol, since the oil globule size of the composition containing propofol is maintained less than the clinically acceptable maximum diameter, even though a large amount of lidocaine, clinically acceptable, is admixed or a relatively long time elapses after the admixing in order to reduce the pain on injection of propofol.

What is claimed is:

1] A parenteral composition comprising propofol, water-immiscible solvent, anionic surfactant, tonicity agent, electrokinetic stabilizer and water.

2] The parenteral composition of claim 1, wherein the elecrokinetic modifier maintains the absolute value of zeta potential of the propofol emulsion at higher than the critical zeta potential.

3] The parenteral composition of claim 2, wherein the electrokinetic stabilizer is pharmaceutically acceptable and injectable, and is selected from the group consisting of a basic amino acid such as lysine, arginine or histidine; a basic compound or a salt such as monoethanolamine, diethanolamine, sodium carbonate, sodium bicarbonate, tromethamine or sodium phosphate; or a mixture thereof.

4] The parenteral composition of claim 3, wherein the electrokinetic stabilizer is present in an amount from 0.005 to 5.0% by weight.

5] The parenteral composition of claim 4, wherein the electrokinetic stabilizer is present in an amount from 0.01 to 0.5% by weight.

6] The parenteral composition of claim 1-5, wherein propofol is present in an amount from 1.0 to 5.0% by weight.

7] The parenteral composition of claim 1-5, wherein the water-immiscible solvent is selected from the group consisting of a vegetable oil such as soybean oil, safflower oil, cottonseed oil, corn oil, sunflower oil, peanut oil, castor oil or olive oil; an ester of a medium chain fatty acid; an ester of a long chain fatty acid; or a mixture thereof.

8] The parenteral composition of claim 7, wherein the water-immiscible solvent is present in an amount from 1.0 to 30.0% by weight

9] The parenteral composition of claim 1-5, wherein the anionic surfactant is selected from the group consisting of a phospholipid such as egg lecithin or soybean lecithin; its derivatives such as phosphatidylcholine, phosphatidylethanolamine, phosphatidylinositol, phosphatidylserine, sphingomyelin, cardiolipin, sulfatide or phosphatidic acid; or a mixture thereof.

10] The parenteral composition of claim 9, wherein the anionic surfactant is present in an amount from 0.2 to 2.0% by weight.

11] The parenteral composition of claim 1-5, wherein the tonicity agent is selected from the group consisting of glycerin, mannitol or sucrose; or a mixture thereof.

12] The parenteral composition of claim 11, wherein the tonicity agent is present in an amount from 0.1 to 3.0% by weight.

13] A method for preparation of a parentral pharmaceutical composition of propofol, which comprises:
a) adding propofol to water-immiscible solvent and heating said solution at 60-85° C. to prepare the oil phase;
b) dispersing anionic surfactant in water for injection, adding tonicity agent and electrokinetic stabilizer to said dispersion, and heating at 60-85° C. to prepare the aqueous phase; and
c) adding the oil phase to the aqueous phase to prepare the emulsion.
